(19) United States
(12) Patent Application Publication
(10) Pub. No.: US 2008/0308571 A1
(43) Pub. Date: Dec. 18, 2008

(54) Adhesive Bandage Dispenser

(76) Inventor: Ehud Rattner, Haifa (IL)

Correspondence Address: WOLF, BLOCK, SHORR AND SOLIS-COHEN LLP, 250 PARK AVENUE, 10TH FLOOR, NEW YORK, NY, 10177, US

(21) Appl. No.: 12/099,492

(22) Filed: Apr. 8, 2008

(30) Foreign Application Priority Data
Apr. 10, 2007 (IL) 182447

(51) Int. Cl. G07F 11/66

(52) U.S. Cl. 221/25

(57) ABSTRACT

The present invention discloses a bandage dispenser for dispensing ready-to-adhere bandages from a cartridge. The cartridge is provided with a plurality of stacked plates accommodated within, and a resilient element beneath a lowermost plate that pushes the stacked plates toward the opening. Adhesive bandage are mounted on the plates with exposed adhesive material. An ejection mechanism ejects an uppermost plate so as to allow a sequential plate with an exposed bandage to move upwardly towards the opening.

### CROSS-REFERENCE TO RELATED APPLICATION

This present invention claims the benefit of earlier IL patent application S.N. 182447 filed on 10 Apr. 2006 by RATTNER Ehud and entitled “Adhesive Bandage Dispenser”.

### FIELD OF THE INVENTION

The present invention relates to bandage dispenser. More particularly, the present invention relates to sterile bandage dispenser for dispensing bandages directly adhered.

### BACKGROUND OF THE INVENTION

Adhesive bandages are the most widely used first aid supplies that are available. The bandages are typically used to decrease prolonged loss of body fluids and stop bleeding by covering and protecting wounds such as cuts, scrape, blisters, bruises and the like. Therefore, bandages are extensively used in hospitals, schools, at home, and basically anywhere.

However, although the bandages are ready to use, for actual adhering the pad on the cut or bruise, the wrapping has to be removed as well as at least two base layers that are adhered onto the adhesive portion. This is a time consuming action that requires some extent of experience and delicacy especially when small bandages are used. The adhesive portions of the tape can be adhered to the fingers of the inexperienced user as well as to be adhered to one another while ruining the bandage.

Most of the bandages are being packed in bulks of individual bandages; however, some dispensers were developed over the years as an answer to a prolonged need. An example is disclosed in U.S. Pat. No. 5,358,140 “Adhesive bandage dispensing system” by Pellegrino. The dispensing system includes an elongated strand of individually sterile-wrapped adhesive bandages and a reusable dispenser for string and dispensing the bandages. Each bandage is contained in a separate sterile compartment of an otherwise continuous wrapping material. Another individual bandage dispenser is described in U.S. Pat. No. 5,271,522 “Individual bandage dispenser” by Ko et al wherein the bandages are adhered to successive bandages along opposite ends of the bandage. The bandages are dispensed from a box between pair of flexible fingers extending into an opening in the top wall of the box.

The solutions given herein as well as other solutions do not provide an answer to the problem of removing the base layers from the adhesive material and therefore merely organize the individual bandages and dispense them individually.

There is still a need to organize the bandages and dispense them individually without the need to handle the exposure of the adhesive portions. There is a need to provide bandages in any size that can be immediately adhered onto the wound without wasting valuable time.

### SUMMARY OF THE INVENTION

It is an object of the present invention to provide adhesive bandage dispenser that is dispensing individual bandage ready for immediate use.

It is another object of the present invention to provide adhesive bandage dispenser that is easy to handle and can be easily used by laypersons.

It is thus provided in accordance with the present invention, An adhesive bandage dispenser comprising:

a cartridge having walls, bottom, and upper opening;

a plurality of stacked plates accommodated within said cartridge;

a resilient element provided within said cartridge beneath a lowermost plate, wherein said resilient element is adapted to push said plurality of stacked plates toward said upper opening;

an adhesive bandage mounted onto each one of said plurality of plates wherein adhesive material of said adhesive bandage is exposed;

a trigger adapted to eject an uppermost plate of said plurality of plates so as to allow a sequential plate to move upwardly towards said upper opening.

Furthermore and in accordance with another preferred embodiment of the present invention, said adhering bandage is adhered onto the plate by adhering strips.

Furthermore and in accordance with another preferred embodiment of the present invention, said adhering material is stronger in adherence characteristics than said adhering strips.

Furthermore and in accordance with another preferred embodiment of the present invention, cartridge is design to correspond the dimensions of said adhering bandage.

Furthermore and in accordance with another preferred embodiment of the present invention, said dimensions are selected from a group of dimensions such as standard, circular, rectangular, square, etc.

Furthermore and in accordance with another preferred embodiment of the present invention, said cartridge is adapted to be incorporated in another device.

Furthermore and in accordance with another preferred embodiment of the present invention, said another device is selected from a group of devices such as shaving devices.

Furthermore and in accordance with another preferred embodiment of the present invention, said resilient element is a spring.

Furthermore and in accordance with another preferred embodiment of the present invention, said cartridge is provided with a protector for protecting said trigger.

Furthermore and in accordance with another preferred embodiment of the present invention, said trigger is adapted to eject the uppermost plate through a rotational movement.

Furthermore and in accordance with another preferred embodiment of the present invention, each plate of said plurality of plates is provided with at least one recess adapted to correspond at least one protrusion in said cartridge that is adapted to prevent the plate that is positioned in said upper opening from being removed from said cartridge and secure the plate in an uppermost position.

Furthermore and in accordance with another preferred embodiment of the present invention, four recesses are provided to correspond four protrusions.

Furthermore and in accordance with another preferred embodiment of the present invention, each plate of said plurality of plates is provided with at least one niche adapted to correspond at least one protrusion in said cartridge that is adapted to prevent withdrawal of the plate inwardly when the plate is positioned in said upper opening.

Furthermore and in accordance with another preferred embodiment of the present invention, four niches are provided to correspond four protrusions.

Furthermore and in accordance with another preferred embodiment of the present invention, the plates are provided with at least one clamp adapted to secure and tight the plate to a plate positioned beneath.

Furthermore and in accordance with another preferred embodiment of the present invention, two clamps are provided on both sides of the plate.

Furthermore and in accordance with another preferred embodiment of the present invention, at least one sliding rail is provided on an upper surface of the plates so as to receive said at least one clamp.

Furthermore and in accordance with another preferred embodiment of the present invention, material is provided between the plates adapted to maintain sterilization of the bandages.

Additionally, and in accordance with another preferred embodiment of the present invention, said material is selected from a group of materials such as paraffin.

### BRIEF DESCRIPTION OF THE DRAWINGS

In order to better understand the present invention and appreciate its practical applications, the following Figures are attached and referenced herein. Like components are denoted by like reference numerals.

It should be noted that the figures are given as examples and preferred embodiments only and in no way limit the scope of the present invention as defined in the appending Description and Claims.

FIG. 1a illustrates an upper view of a bandage dispenser in accordance with a preferred embodiment of the present invention.

FIG. 1b illustrates an A-A cross sectional view of the bandage dispenser shown in FIG. 1a.

FIGS. 2a,b illustrate a side cross sectional view and an upper view, respectively, of the cartridge and spring shown in FIG. 1.

FIGS. 3a,b illustrate side view and upper view, respectively, of a side panel of the cartridge as shown in FIG. 1.

FIGS. 4a,b illustrate a middle plate in accordance with a preferred embodiment of the present invention, side view and upper view.

FIGS. 5a,b illustrate an upper plate in accordance with a preferred embodiment of the present invention, side view and upper view.

FIGS. 6a,b illustrate a bottom plate in accordance with a preferred embodiment of the present invention, side view and upper view.

FIGS. 7a,b illustrate a adhesive bandage in accordance with a preferred embodiment of the present invention, side view and upper view.

FIG. 8 illustrates a razor incorporated with an adhesive bandage dispenser in accordance with a preferred embodiment of the present invention.

FIG. 9 illustrates the razor shown in FIG. 8, in a disengaged state.

FIGS. 10a-c illustrate an inner structure of a razor's handle in accordance with a preferred embodiment of the present invention.

FIG. 11 illustrates a razor device in accordance with another preferred embodiment of the present invention.

FIGS. 12a, b illustrate an upper plate in accordance with another preferred embodiment of the present invention, side view and upper view.

FIGS. 13a, b Illustrate side view and upper view, respectively, of a side panel of a cartridge in accordance with another preferred embodiment of the present invention.

FIGS. 14a-d illustrate a middle plate in accordance with a preferred embodiment of the present invention, side view, upper view, cross sectional view, and isometric view.

FIGS. 15a, b illustrate isometric views of a bandage dispenser in accordance with another preferred embodiment of the present invention, partially exposing inner areas.

FIG. 16 illustrates the bandage dispenser shown in FIG. 15 provided with the plate in accordance with another preferred embodiment of the present invention.

FIG. 17 illustrates different views of the plate shown in FIG. 16.

FIG. 18 illustrates different views of a bandage dispenser in accordance with yet another embodiment of the present invention.

### DETAILED DESCRIPTION OF THE INVENTION

The present invention provides a unique and novel adhesive bandage dispenser that is adapted to dispense individual bandages ready to be immediately adhered onto the wound. According to the concept of the present invention, the adhesive bandage is dispensed with the portions of adhering materials and pad exposed to be immediately adhered onto the wound without the need to take off the wrapping and to peel off base layers.

According to one concept of the present invention, the bandages are individually placed on plates that are organized one on top of the other in a cartridge. A resilient member such as a spring pushes the plates outwardly of the cartridge while revealing the uppermost bandage with the pad and adhesive portions outwardly exposed. The user adhere the bandage in a very simple and straightforward manner onto the wound.

According to another concept of the present invention, the cartridge is adapted to be incorporated within a handle, as an example, of a device that is cut-prone such as a shaving device.

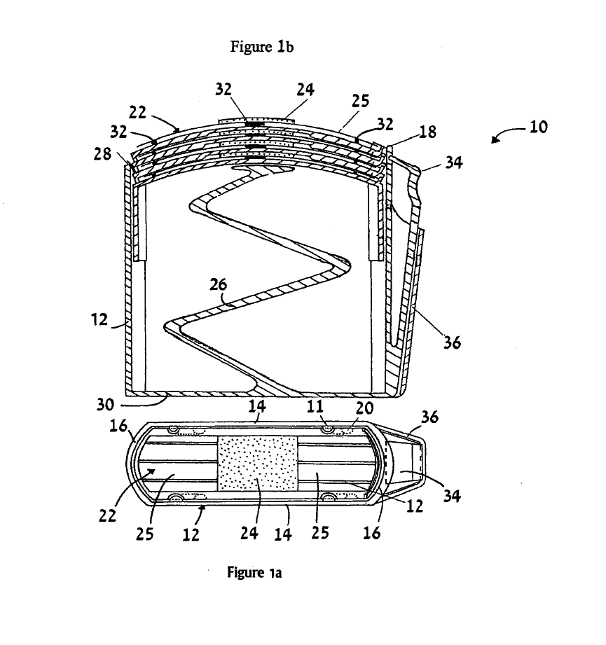

Reference is made to FIGS. 1a and b illustrating an upper view and cross sectional view, respectively, of a bandage dispenser in accordance with a preferred embodiment of the present invention. Dispenser 10 comprises a cartridge 12 having side panels 14, side walls 16, bottom side 30, and an upper opening 28 wherein in the embodiment shown herein as an example, the side panels and side walls are constructed to accommodate a standard size elongated bandage.

A plurality of plates 18 are organized one on top of the other within cartridge 12 while each plate is provided with recess 20 shown in FIG. 1a in dash line and will be elaborated on herein after. The uppermost plate is placed in and blocks upper opening 28. Each plate is provided with an adhering bandage 22 that is placed onto plate 18 and adhered to it by adhering strips 32. The adhesive bandage is provided with a pad 24 and adhesive material 25 exposed upwardly.

It is important to notice that adhering strips 32 are provided with adhering material having adhering properties that are less strong than the adhesive material that is exposed upwardly on the surface of the bandage and is adapted to be adhered to the skin of the user. The reason for that is that when the user adhere the bandage onto his skin, the removal of the bandage off the plate will be very easy and there will be almost no resistance to adhering the bandage onto the skin.

A spring 26 that is provided within cartridge 12 and beneath plurality of plates 18 pushes at all times plates 18 upwardly towards upper opening 28 of the cartridge so that as long as there are plates in the cartridge, the uppermost one will be positioned in upper opening 28.

When the user places the adhesive bandage onto a wound, the uppermost plate serves as a protector to the adhesive bandage that is positioned beneath it on the sequential plate. In order to use an additional bandage, the uppermost plate is removed using a trigger 34 that is being pushed by the user's finger so as to push off the plate. Trigger 34 is provided with a protector 36 that is preferably incorporated within cartridge 12.

In order to better understand the functionality of the elements of the dispenser of the present invention, the parts are shown one by the other.

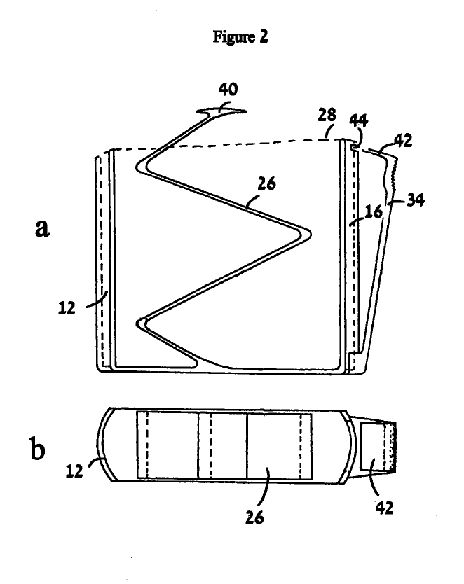

Reference is now made to FIGS. 2a and b illustrating a side cross sectional view and an upper view of the cartridge and spring shown in FIG. 1. Spring 26 is better seen in FIG. 2a with a default state that is markedly protruding beyond upper opening 28 so that the spring has enough force to push the plates (are not shown in this figure) upwardly. Spring 26 is provided with a supporting base 40 adapted to establish a supporting area to the lowermost plate.

Optionally, any resilient element or coiled spring can be provided within cartridge 12 that is adapted to push the plates upwardly.

Trigger 34 is shown to have a pin 42 that can pass through a hole 44 in side wall 16 of the cartridge. Pin 42 is adapted to remove the uppermost plate that is provided at any time in the cartridge.

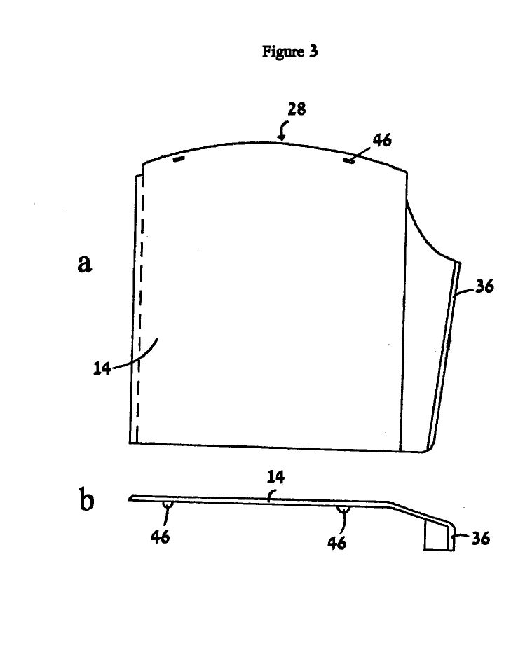

Reference is now made to FIGS. 3 a and b illustrating a side view and an upper view of a side panel of the cartridge as shown in FIG. 1. Side panel 14 is constructed to form a housing while protrusions 46 are provided beneath upper opening 28 line directed to be in the interior of the cartridge. Optionally, and as shown in the present embodiment, four protrusions are provided on four corners of opening 28. Plurality of plates 18 comprises basically three types of plates. Middle plates, upper plate, and bottom plate.

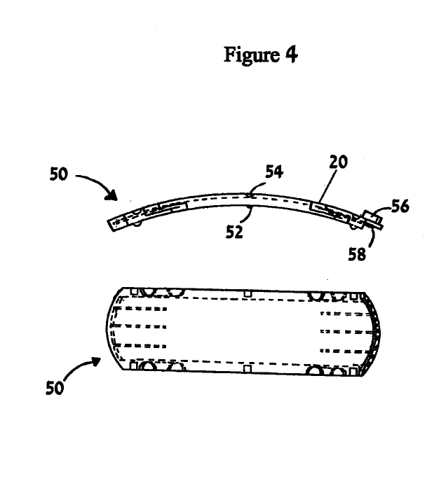

Reference is now made to FIGS. 4 a and b illustrating a middle plate in accordance with a preferred embodiment of the present invention. Middle plate 50 is preferably constructed as a curved plate provided with four recesses 20 that are provided on the sides of the plate so as to correspond to the four protrusions 46 of the cartridge. When a plate is reaching an uppermost position in upper opening 28, the protrusions are inserted within the recesses in the plate so as to fix the plate in this upper position.

In order to separate and keep a predetermined and controlled distance between sequential plates 18, a separation leg 52 is provided in the bottom surface of plate 50 and a mounting recess 54 is provided in the upper surface of the plate so as to allow separation leg 52 to be in association with recess 54.

A side protrusion 56 is provided on the upper surface of plate 50 and a corresponding recess 58 is provided on the lower surface of the plate, both on one side of the plate that faces the trigger that eject the upper plate (the trigger is not shown in this figure). Protrusion 56 is adapted to maintain the distance between two consequent plates since the plates are preferably curved. The protrusion also facilitate in maintaining the sterilization of the bandage the is adhered onto the plate.

The upper surface of middle plate 50 is provided with fixation ribs adapted to maintain the bandage that is adapted to be mounted in the plate.

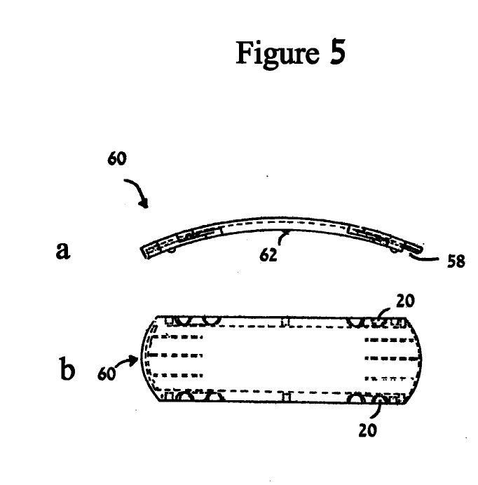

Reference is now made to FIG. 5 illustrating an upper plate in accordance with a preferred embodiment of the present invention, side view and upper view. Basically, upper plate 60 is similar to middle plate 50, However, there is only separation leg 62 in the lower surface of the plate while an mounting recess in the supper surface is eliminated. Similarly, a recess 58 is provided on the lower surface of the plate and since the plate is the uppermost one, a protrusion is eliminated.

Recesses 20 are also provided similarly to the recesses in the middle plate.

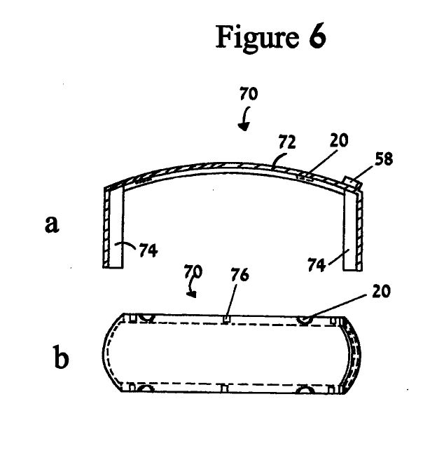

Reference is now made to FIG. 6 a and b, illustrating a bottom plate in accordance with a preferred embodiment of the present invention, side cross sectional view and upper view. Bottom plate 70 comprises an upper curves surface 72 and stabilizers 74 that are adapted to provide stability to the plate and to the plates that are stacked onto the bottom plate. Similarly to all other plates, upper surface 72 of bottom plate 70 is provided with recesses 20 adapted to associate with protrusions 46 provided on the side panel of the cartridge. A mounting recess 76 is adapted to receive a separating leg from the middle plate that is mounted on top of bottom plate 70. A protrusion 58 is provided on the upper surface of the plate as explained herein before. The lower recess is eliminated since there is no need for that in the lowermost plate.

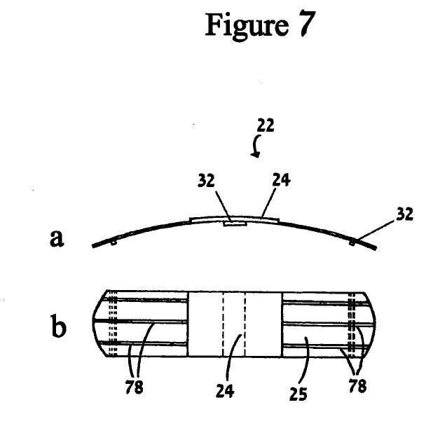

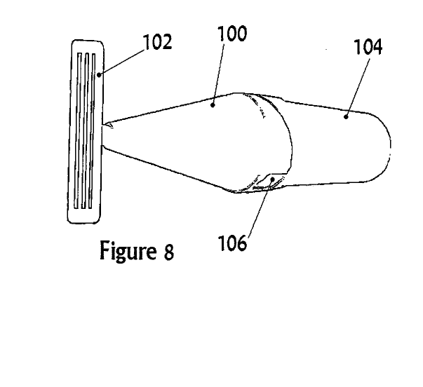

Reference is now made to FIG. 7 a and b illustrating a adhesive bandage in accordance with a preferred embodiment of the present invention, side view and upper view. Bandage 22 is provided with a pad 24 similar to pads that are known in the art. The upper surface of adhesive bandage 22 is similar to standard adhesive bandages, however, the lower surface is provided with adhering strips 32 adapted to adhere the bandage to the plate onto which the adhesive bandage is mounted on.

Optionally, adhesive material 25 is provided on two wings of the bandage and non-adhering strips 78 are provided on the wings surface in order to avoid curling of the bandage.

As mentioned herein before, cartridges of adhesive bandages dispensers can be constructed in any size and may be also incorporated within another device such as a shaving device—a razor.

Reference is now made to FIG. 8 illustrating a razor incorporated with an adhesive bandage dispenser in accordance with a preferred embodiment of the present invention. A razor device 100 is provided with a razor 102 and a dispenser 104 for adhesive bandages. Razors are devices that are cut-prone and people that use them are being cut frequently. Many archaic methods are being used by the users in order to stop the bleeding and cover the wound such as piece of paper that is adhered to the wound, a cotton ball is being put on the wound etc. In accordance with a preferred embodiment of the present invention, a dispenser of adhesive bandages is incorporated within the handle of the razor so that when the user is having a cut, he immediately opens the dispenser to cover the wound and stop the bleeding.

Razor device 100 is further provided with a releasing button 106 adapted to allow release of the connection between the razor and the dispenser so as to expose the bandage.

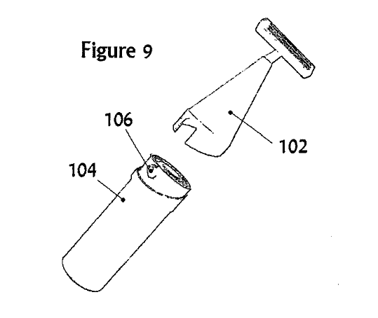

Reference is now made to FIG. 9 illustrating the razor shown in FIG. 8, in a disengaged state. When the user wishes to put a bandage onto a cut, he uses button 106 in order to disengage the two portions of the razor and to expose a bandage 108 that can be directly adhered onto the cut.

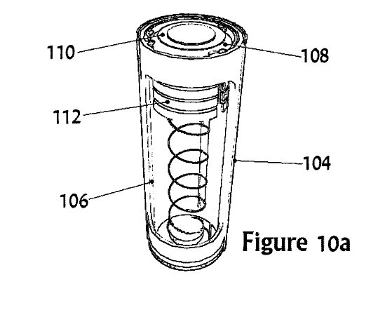

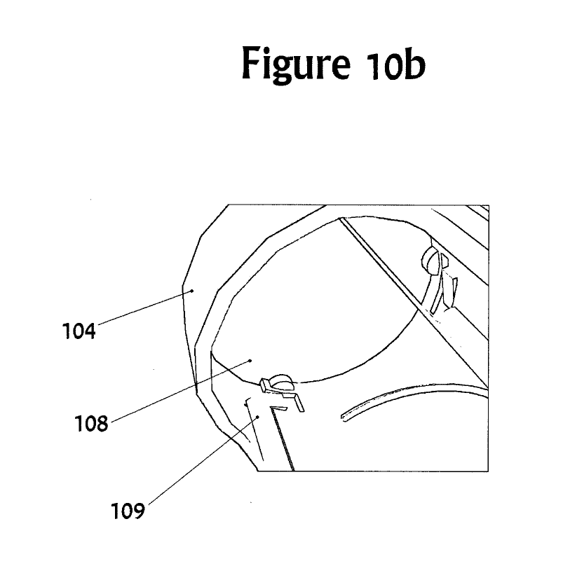

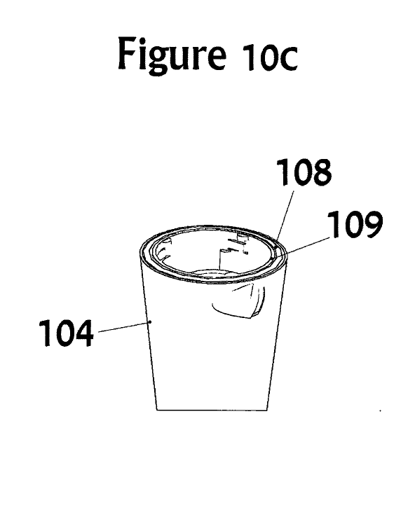

Reference is now made to FIGS. 10a-c illustrating an inner structure of a razor's handle in accordance with a preferred embodiment of the present invention. Basically, the mechanism is similar to the mechanism shown herein before. The inner structure of the razor's handle 104 is provided with a spring 106 adapted to force the plurality of bandages to be pushed toward an upper opening 108 of the handle. Each bandage 110 is being adhered on a plate 112 wherein a plurality of plates is provided in a stacked manner within the handle of the razor. Removal of the razor portion 102 off the handle, in a rotational movement, will push a trigger 109 so as to eject the uppermost plate from upper opening 108 while exposing the bandage that is beneath it and mounted on the sequential lower plate. Since the handle of the razor in this case is circular, the rotational movement is appropriate to eject the uppermost plate of the cartridge, however, other mechanisms can be applies without limiting the scope of the present invention.

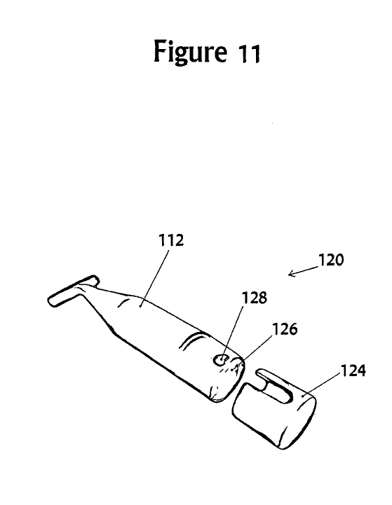

Reference is now made to FIG. 11 illustrating a razor device in accordance with another preferred embodiment of the present invention. Razor device 120 is provided with a razor 122 and a handle 124 having a cover 124 in the far end of the handle. In this case, the razor and the handle are engaged at all times and cannot be disengaged; however, the removal of cover 124 from the handle exposes a bandage 126 having adhering material exposed to be immediately adhered onto the would. Button 128 act as a trigger so that upon removal of cover 124 the upper plate that is covering and protecting and bandage beneath the plate is being removed so as to expose the bandage.

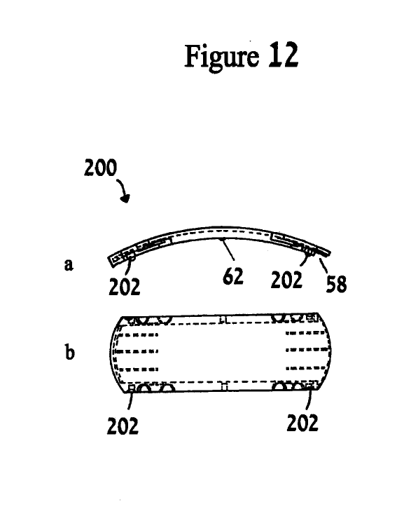

Reference is now made to FIGS. 12 a and b illustrating an upper plate in accordance with another preferred embodiment of the present invention, side view and upper view. This embodiment is designed to prevent possible withdrawal of the plates inwardly in the direction of the cartridge when the adhesive bandage is being adhered onto the skin of the user. Basically, plate 200 is similar to plate 60 however, stoppers 202 are provided preferably on four sides of the plate. Stoppers 202 are niches to which corresponding protrusions are adapted to be inserted in a releasable manner.

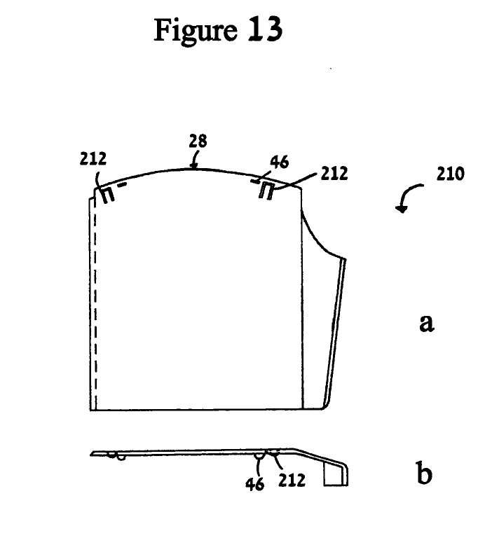

Reference is now made to FIGS. 13 a and b illustrating side view and upper view, respectively, of a side panel of a cartridge in accordance with another preferred embodiment of the present invention. Basically, panel 210 is similar to panel 14 shown in FIGS. 3 a and b, however, additional protrusions 212 are provided in the upper zone of the panel, near the upper opening 28 of the cartridge. Protrusions 212 correspond to niches 202 that are shown in FIGS. 12 a and b so as to prevent upper plate 200 from withdrawing inwardly when force is applied onto the plate.

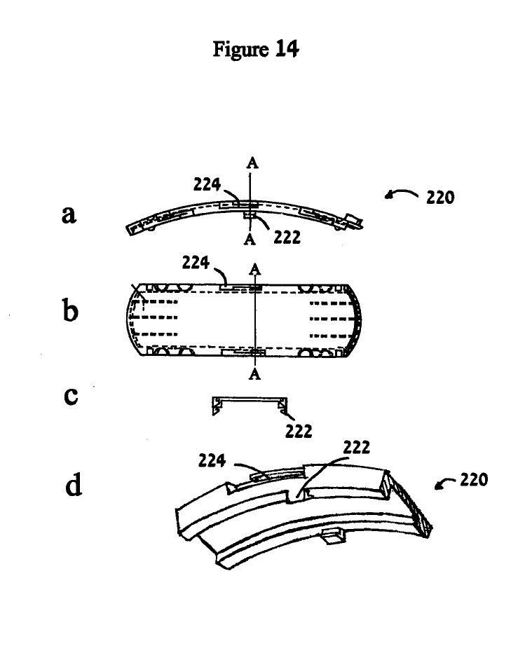

Reference is now made to FIGS. 14 a-d illustrating a middle plate in accordance with a preferred embodiment of the present invention, side view, upper view, cross sectional view, and isometric view. The middle plates are most of the plates that are provided in the cartridge. As mentioned herein before, the middle plates are moving upwardly towards the upper opening of the cartridge as long as the product is consumed. The plate that is provided in the uppermost position is ejected and the one beneath it is exposed with the new adhering bandage. In order to assure the sterilization of the bandages that are adhered onto the plates, the plates are tight to each other. This embodiment is provided with an additional means of tightening two consequent plates together. Middle plate 220 is provided with two clamps 222 on both sides of the plate that are adapted to be secured to the plate positioned beneath. Clamps 222 are standard clamps that are adapted to release the plate from its secured place when the plate is being ejected. The upper surface of plate 220 is provided with sliding rail 224 through which the plates are being secured to one another. An isometric view of middle plate 220 is depicting both features.

It should be noted that paraffin is being added between the plates so as to increase the sealing features between the consequent plates so as to assure sterilization. Other materials can be used.

It should be emphasized that there is no need to remove the bandage off the plate using the user's fingers. The bandage is being adhered directly onto the wound.

In accordance with another aspect of the present invention, there is provided a mechanism that preserves that sterilization of the inner plates even better then the previous shown mechanism. The basic idea of the bandage dispenser is kept intact.

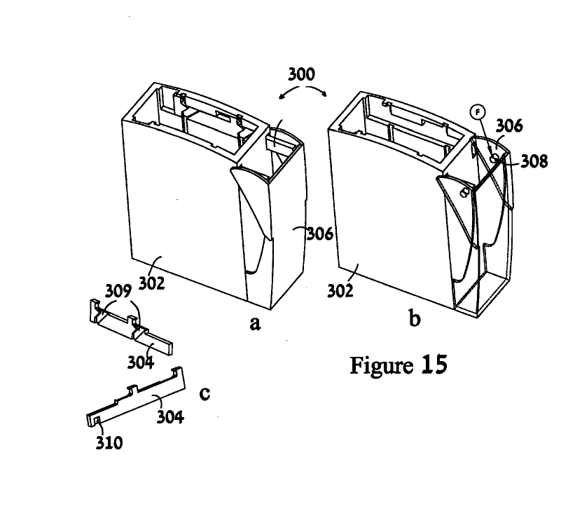

Reference is now made to FIG. 15 illustrating isometric views of a bandage dispenser in accordance with another preferred embodiment of the present invention, partially exposing inner areas. Shown in FIG. 15a, a dispenser 300 that is provided with a cartridge 302 having walls and an upper opening allowing a bandage to be upwardly exposed as shown herein before. Cartridge 302 is provided with a trigger 304 and protector 306 capable of dispensing the plates (the plates are not shown in FIG. 15a and b). Trigger 304 is provided in the upper portion of cartridge 302 and establishes the mechanism that ejects the plates. Trigger 304 itself is shown in the figure (FIG. 15c). One side of trigger 304 is provided with guiding indentation 309 for guiding the uppermost plate to ejection and the other side is provided with a recess 310 adapted to receive a pin 308 that is provided to protector 306. Trigger 304 is moving inwardly towards the interior of cartridge 302 in order to eject the plate by the movement of protector 306 with pin 308 that guides trigger 304. The pin is clearly seen in FIG. 15b.

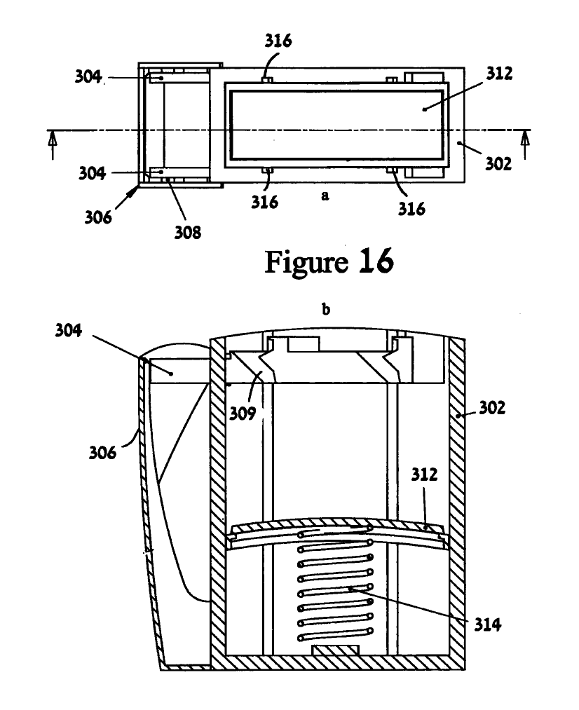

Reference is now made to FIG. 16 illustrating the bandage dispenser shown in FIG. 15 provided with the plate in accordance with another preferred embodiment of the present invention. FIG. 16a illustrates an upper view of the bandage dispenser with a plate 312 provided within cartridge 302. As shown herein before, a spring 314 is associated with the lower portion of cartridge 302 pushing plate 312 upwardly as a default as shown in a cross sectional view of the cartridge in FIG. 16b. Plate 312 is provided with protrusions 316 adapted to be guided towards ejection of the plate by indentation 309 of trigger 304.

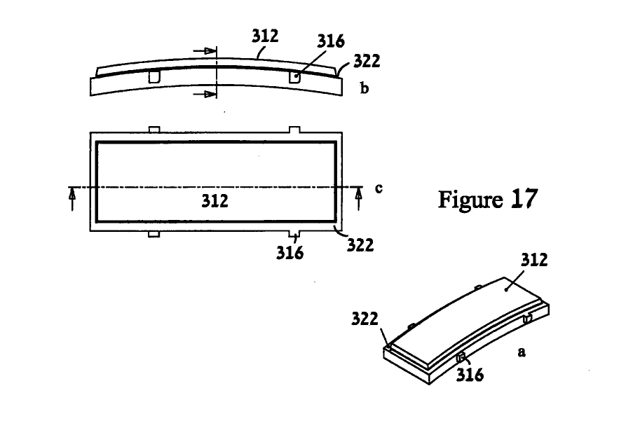

Reference is now made to FIG. 17 illustrating different views of the plate shown in FIG. 16. An isometric view shown in FIG. 16a, protrusions 316 are clearly shown to be provided in the sides of plate 312. a surface 320 is provided to adhere the bandage as shown in the previous embodiment and a shoulder 322 is provided for exposing only the upper surface of the plate outwardly of the cartridge so that the lower portion of covering the opening of the cartridge in a manner that best preserves sterilization of the lower plates provided with the bandages.

The movement of the trigger is forcing the uppermost plate to be ejected of the cartridge and expose a new and sterile bandage.

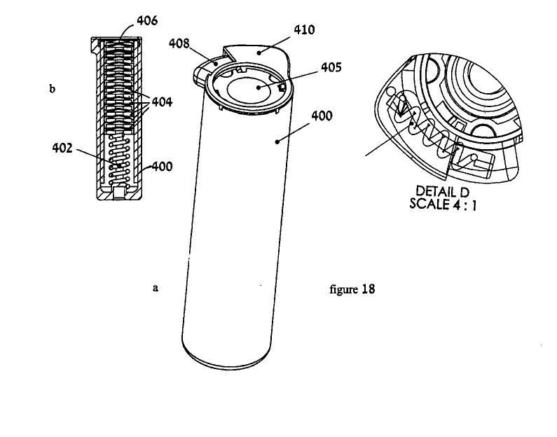

Reference is now made to FIG. 18 illustrating different views of a bandage dispenser in accordance with yet another embodiment of the present invention. FIG. 18a illustrates a bandage dispenser 400 having a rounded profile. In the cross sectional view in FIG. 18b, a spring 402 is shown to push a plurality of plates 404 upwardly towards the opening 406 of cartridge 400. Back to FIG. 18a, the upper portion is shown to have a mechanism that ejects outwardly the uppermost plate 405.

The ejection mechanism comprises a constant member 410 that is outwardly protruded in the upper portion of cartridge and a movable portion 408 that is mounted on the upper portion of the cartridge. Movable portion 408 is adapted to move in respect with member 410 wherein a spring 412 is provided in a niche between the portions (Shown in FIG. 18c). The spring is adapted to maintain portion 408 in a predetermined position relative to member 410 and to forcibly move relative to member 410 in order to eject uppermost plate 405. The plates are stacked together so as to establish sterile conditions within the cartridge.

It should be clear that the description of the embodiments and attached Figures set forth in this specification serves only for a better understanding of the invention, without limiting its scope as covered by the following Claims.

It should also be clear that a person skilled in the art, after reading the present specification can make adjustments or amendments to the attached Figures and above described embodiments that would still be covered by the following Claims.

1. An adhesive bandage dispenser comprising:
a cartridge having walls, bottom, and upper opening;
a plurality of stacked plates accommodated within said cartridge;
a resilient element provided within said cartridge beneath a lowermost plate, wherein said resilient element is adapted to push said plurality of stacked plates toward said upper opening;
an adhesive bandage mounted onto each one of said plurality of plates wherein adhesive material of said adhesive bandage is exposed;
an ejection mechanism adapted to eject an uppermost plate of said plurality of plates so as to allow a sequential plate to move upwardly towards said upper opening.

2. The dispenser as claimed in claim 1, wherein said adhering bandage is adhered onto the plate by adhering strips.

3. The dispenser as claimed in claim 2, wherein said adhering material is stronger in adherence characteristics than said adhering strips.

4. The dispenser as claimed in claim 1, wherein cartridge is design to correspond the dimensions of said adhering bandage.

5. The dispenser as claimed in claim 4, wherein said dimensions are selected from a group of dimensions such as standard, circular, rectangular, square, etc.

6. The dispenser as claimed in claim 1, wherein said cartridge is adapted to be incorporated in another device.

7. The dispenser as claimed in claim 6, wherein said another device is selected from a group of devices such as shaving devices.

8. The dispenser as claimed in claim 1, wherein said resilient element is a spring.

9. The dispenser as claimed in claim 1, wherein said ejection mechanism is provided with a trigger.

10. The dispenser as claimed in claim 9, wherein said cartridge is further provided with a protector for protecting said trigger.

11. The dispenser as claimed in claim 9, wherein said trigger is adapted to eject the uppermost plate through a rotational movement.

12. The dispenser as claimed in claim 1, wherein said ejection mechanism is provided with a trigger that is moving in a rotational movement and is maintained in a default position using a resilient member.

13. The dispenser as claimed in claim 12, wherein said resilient member is a spring.

14. The dispenser as claimed in claim 1, wherein each plate of said plurality of plates is provided with at least one recess adapted to correspond at least one protrusion in said cartridge that is adapted to prevent the plate that is positioned in said upper opening from being removed from said cartridge and secure the plate in an uppermost position.

15. The dispenser as claimed in claim 14, wherein four recesses are provided to correspond four protrusions.

16. The dispenser as claimed in claim 1, wherein each plate of said plurality of plates is provided with at least one niche adapted to correspond at least one protrusion in said cartridge that is adapted to prevent withdrawal of the plate inwardly when the plate is positioned in said upper opening.

17. The dispenser as claimed in claim 16, wherein four niches are provided to correspond four protrusions.

18. The dispenser as claimed in claim 1, wherein the plates are provided with at least one clamp adapted to secure and tight the plate to a plate positioned beneath.

19. The dispenser as claimed in claim 18, wherein two clamps are provided on both sides of the plate.

20. The dispenser as claimed in claim 18, wherein at least one sliding rail is provided on an upper surface of the plates so as to receive said at least one clamp.

21. The dispenser as claimed in claim 1, wherein said ejection mechanism is provided with a trigger having indentation capable of guiding the uppermost plate outwardly of the opening of the cartridge.

22. The dispenser as claimed in claim 21, wherein the plates are provided with protrusions adapted to be guided through said indentation.

23. The dispenser as claimed in claim 1, wherein material is provided between the plates adapted to maintain sterilization of the bandages.

24. The dispenser as claimed in claim 21, wherein said material is selected from a group of materials such as paraffin.
